(19) United States
(12) Patent Application Publication
(10) Pub. No.: US 2005/0169949 A1
(43) Pub. Date: Aug. 4, 2005

(54) Makeup composition for keratin materials

(76) Inventors: Valerie De La Poterie, (US); Karl Pays, Saint-Maurice (FR); Yohann Bichon, Maisons-Alfort (FR); Therese Daubige, Beaumont-En-Veron (FR); Patrice Styczen, Gif Sur Yvette (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/911,671

(22) Filed: Aug. 5, 2004

Related U.S. Application Data
(60) Provisional application No. 60/495,886, filed on Aug. 19, 2003.

(30) Foreign Application Priority Data
Aug. 6, 2003 (FR) 03 09711

(51) Int. Cl. A61K 7/00

(52) U.S. Cl. 424/401

(57) ABSTRACT

The present disclosure relates to a cosmetic composition for making up keratin fibers, comprising a non-aqueous solvent medium, at least one wax in an amount of greater than 3% by weight, relative to the total weight of the composition, at least one polymer that is soluble in the solvent medium and has at least one crystallizable portion, and water and/or at least one water-soluble solvent, whererin the water and/or at least one water-soluble solvent is present in a total amount of less than or equal to 20% by weight, relative to the total weight of the composition, and the composition has a solids content of greater than 45% by weight relative to the total weight of the composition. Also disclosed herein are processes for preparing such compositions and methods of using same.

This application claims benefit of U.S. Provisional Application No. 60/495,886, filed Aug. 19, 2003, which is herein incorporated by reference.

The present disclosure relates to a make-up composition for keratin materials, such as keratin fibers, including the eyelashes, the eyebrows and the hair. The present disclosure also relates to a method of making such a composition, as well as a method of using such a composition for making-up keratin materials.

According to the present disclosure, the composition as disclosed herein may be in the form of mascara, eyebrow products, eyeliner or hair makeup products. In one embodiment of the present disclosure the composition is a mascara. In another embodiment of the present discosure the composition as disclosed herein comprises makeup compositions, compositions to be applied over or under a makeup, also known, respectively, as a “top coat” or a “base coat”, or compositions for treating keratin fibers, such as the eyelashes.

In general, compositions for making up keratin fibers, for instance the eyelashes, may comprise at least one wax or a mixture of waxes dispersed in a liquid phase. Variation of the amount of wax and of the other non-volatile ingredients, reflected by the solids content of the composition, is commonly how the desired application specificities for the compositions are adjusted, for instance their fluidity, their covering power and/or their curling power, and also their thickening power (also known as charging power or makeup power).

There are two main types of mascara formulation in practice. First, water-based mascaras, known as “cream mascaras,” which are in the form of a wax-in-water emulsion, and, second, anhydrous mascaras or mascaras with a low content of water and/or of water-soluble solvents, known as “waterproof mascaras”, which are formulated in the form of a dispersion of waxes in non-aqueous solvents. It should be noted, however, that certain mascaras in the form of wax-in-water emulsions are also termed “waterproof”. Their water resistance basically results from the presence of a large amount of latex in their composition. However, they are also characterized by a low solids content and thus can have very little makeup power.

Thus, the present disclosure relates, for example, to the field of keratin fiber makeup compositions comprising a low content of water and/or of water-soluble solvents, known as “waterproof mascaras,” which are in the form of a dispersion of at least one wax in at least one non-aqueous solvent.

Conventionally, such compositions can have a solids content ranging from 15% to 45% by weight, relative to the total weight of the composition. As stated previously, this solids content range can lead to unsatisfactory makeup results. However, increasing the solids content beyond this value, can lead to the problem of lack of fluidity, wherein the makeup composition can become too thick to apply and also may no longer have the deformability required for uniform application over the entire surface of the eyelashes. Moreover, microscopic observation shows that, in this type of composition, the wax particles may be in the form of aggregates.

Accordingly, the present disclosure relates to, for example, a makeup or care composition for keratin fibers that has a high solids content, which can make it possible, for instance, to obtain a makeup result that is thicker than that obtained with traditional “waterproof” compositions, while at the same time capable of maintaining a consistency that is compatible with the intended makeup use.

Further, the present disclosure relates to a composition comprising a specific polymer, such as a polymer that is both soluble in the non-aqueous solvent medium of the composition and endowed with a crystallizable portion. This type of polymer makes it possible to significantly improve the dispersion of the wax particles in the liquid phase.

Thus, one aspect of the present disclosure relates to, for example, a cosmetic makeup composition for keratin fibers, comprising:
(i) a non-aqueous solvent medium,
(ii) at least one wax in an amount greater than 3% by weight, relative to the total weight of the composition,
(iii) at least one polymer that is soluble in the solvent medium and that has at least one crystallizable portion, and
(iv) water and/or at least one water-soluble solvent, wherein the water and/or the at least one water-soluble solvent are present in a total amount of less than or equal to 20% by weight, relative to the total weight of the composition, and wherein
   the composition has a solids content of greater than 45% by weight, relative to the total weight of the composition.

Another aspect of the present disclosure, is a process for preparing a composition as defined above, comprising at least the continuous blending of at least one wax and of at least one polymer that is soluble in a non-aqueous solvent and that has a crystallizable portion, by passing from a temperature above the melting point of the at least one wax to room temperature via continuous cooling.

The present disclosure also relates to a process for preparing a composition as defined above, comprising at least one step of dispersing at least one wax in the form of particles ranging from 0.5 μm to 30 μm, for instance, ranging from 1 to 20 μm in size, in a mixture comprising at least one non-aqueous solvent and at least one polymer that is soluble in the at least one non-aqueous solvent and that has at least one crystallizable portion, the mixture being at a temperature below the melting point of the at least one wax in particle form.

The present disclosure futher relates to the use of at least one polymer that is soluble in a non-aqueous solvent and that has at least one crystallizable portion combined with at least one wax in the form of particles ranging from 0.5 μm to 30 μm, for instance, from 1 to 20 μm in size, for the preparation of a makeup composition for keratin fibers, such as a composition as disclosed herein.

The present disclosure still further relates to a process for making up keratin fibers, wherein a composition as defined above or as obtained via one of the processes as defined above is applied to keratin fibers, for instance, the eyelashes.

The compositions as disclosed herein may also have a higher drying speed than standard waterproof compositions, which makes it possible to reduce the time required to perform the makeup process and can reduce the risk of transferring makeup from the eyelashes onto the adjacent eyelid. This fast drying time can also make it possible, where appropriate, to be able to apply several layers of the composition in a satisfactory time and thus to further reinforce the thickening effect of the makeup obtained with these compositions.

Description of the Solids

As disclosed herein, the term “solids content” refers to the content of non-volatile matter.

This amount of solids, commonly referred to as the “dry extract” or its abbreviated form DE, of the compositions according to the present disclosure is measured by heating the sample with infrared rays with a wavelength ranging from 2 μm to 3.5 μm. The substances comprised in the compositions that have a high vapour pressure evaporate under the effect of this radiation. Measurement of the weight loss of the sample makes it possible to determine the “dry extract” of the composition. These measurements are performed using an “LP16®” commercial infrared desiccator from Mettler. This technique is fully described in the machine documentation supplied by Mettler.

The measuring protocol is as follows:

A 1 g sample of the composition is spread onto a metal crucible. After placing the crucible in the desiccator, it is subjected to a nominal temperature of 120° C. for 1 hour. The wet mass of the sample, which corresponds to the initial mass, and the dry mass of the sample, which corresponds to the mass after exposure to the radiation, are measured using a precision balance.

The solids content is calculated in the following manner:
Dry extract=100×(dry mass/wet mass).

The values measured using the protocol described above may differ from the corresponding theoretical values by plus or minus 1%.

The compositions as disclosed herein comprise a solids content of greater than 45% by weight, for example, ranging from 46% to 80%, such as from 48% to 70% and from 50% to 65% by weight, relative to the total weight of the composition.

Polymers that are Soluble in the Non-Aqueous Solvent Medium and that have at Least One Crystallizable Portion

The composition as disclosed herein comprises at least one polymer that is soluble in the non-aqueous solvent medium and that has at least one crystallizable portion.

As used herein, the expression “polymer that is soluble in the said non-aqueous solvent medium” means a polymer which, when introduced alone in a solids content at least greater than 0.01% by weight and for an amount corresponding to that envisaged for the desired final composition, is soluble in the non-aqueous solvent medium at room temperature, generally of about 25° C., and under atmospheric pressure (750 mmHg, i.e. 10⁵ Pa).

For the purposes of the present disclosure, the term “polymer” means a compound comprising at least two repeating units, for example at least three repeating units, such as at least ten repeating units, or at least fifteen repeating units. The polymers that may be used in accordance with the present disclosure are generally comprised of at least two repeating units of different nature (copolymer). The polymers used as disclosed herein are generally of synthetic origin and can have molar masses ranging from 200 to 1,000,000 g/mol, for instance from 500 to 500,000 g/mol such as from 1,000 to 300,000 g/mol.

For further example, the polymers that may be used according to the present disclosure are copolymers that are dissolved and non-crystalline in the medium at room temperature and comprise at least one crystallizable portion denoted A and at least one “amorphous” non-crystallizable portion, denoted B.

As a result of this specific structure, these polymers can have both affinity for waxes by virtue of the portion A and affinity for the solvent by virtue of the portion B, and thus participate efficiently in this respect in dispersing the waxes in the non-aqueous solvent medium.

The crystallizable portion of the polymers used as disclosed herein can be present in an amount of at least 5%, such as at least 10% and up to 50%, and for further instance, from 30% to 50% by weight, relative to the total weight of each polymer.

The crystallizable portion A of a copolymer as disclosed herein may comprise a pendent chain linked to the skeleton of the polymer and/or a block directly incorporated into the skeleton and/or at least one end chain. These copolymers may be of any chemical structure: random, block or grafted copolymers and/or dendrimers.

Similarly, the amorphous portion of the copolymers as disclosed herein may feature a pendent chain linked to the skeleton of the copolymer and/or a block directly incorporated into this skeleton and/or at least one end chain.

For the purposes of the present disclosure, the following terms or expressions have the meanings given hereinbelow:
“crystallizable portion A” means a sequence of at least 5 repeating units wherein if the homopolymer corresponding to this repeating unit is considered, it would be characterized by a degree of crystallinity of greater than 30%,
“amorphous portion B” means a sequence of at least 5 repeating units wherein if the homopolymer corresponding to this repeating unit is considered, it would be characterized by a degree of crystallinity of less than 5%, or even zero,
“block incorporated into the skeleton” means a group of atoms comprising the repetition of a monomer unit, forming part of the main chain of the polymer,
“pendent chain or side group” means a group of atoms forming a branch on the polymer skeleton, and
“end chain” means a group of atoms located on at least one of the ends of the skeleton.

a) Random Copolymers

Random copolymers are for example, polymers with crystallizable pendent chains, which comprise units resulting from the polymerization of at least two monomers, at least one of which has a crystallizable hydrophobic side chain known as X that may be represented by formula I: 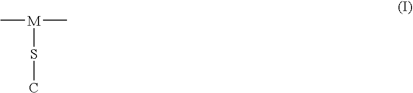
wherein M comprises an atom of the polymer skeleton, S comprises a spacer and C comprises a crystallizable group.

The crystallizable chains “—S—C” may be aliphatic or aromatic, and optionally fluorinated or perfluorinated. For example, “S” can be chosen from (CH₂)ₙ, (CH₂CH₂O)ₙ, and (CH₂O), which may be linear, branched, or cyclic, wherein n is an integer ranging from 0 to 22. For instance, “S” may be a linear group. In another instance, “S” and “C” may be different.

When the crystallizable chains “—S—C” are hydrocarbon-based aliphatic chains, they comprise hydrocarbon-based alkyl chains comprising from 11 to 40 carbon atoms, for instance, not more than 24 carbon atoms. For example, they may be aliphatic chains or alkyl chains comprising at least 12 carbon atoms, such as C₁₄-C₂₄ alkyl chains. When “—S—C” are fluoroalkyl or perfluoroalkyl chains, they comprise at least 6 fluorinated carbon atoms, such as at least 11 carbon atoms, wherein at least 6 of the carbon atoms are fluorinated.

As examples of polymers comprising at least one crystallizable pendent chain, non-limiting mention may be made of those comprising units resulting from the polymerization of at least one the following monomers: (meth)acrylates of saturated alkyls with the alkyl group ranging from C₁₄-C₂₄; perfluoroalkyl(meth)acrylates with a C₁₂-C₁₅ perfluoroalkyl group; N-alkyl(meth)acrylamides with the alkyl group ranging from C₁₂ to C₂₄ optionally with a fluorine atom; vinyl or allyl esters comprising alkyl or perfluoro(alkyl) chains with the alkyl group ranging from C₁₂ to C₂₄ with at least 6 fluorine atoms per perfluoroalkyl chain; vinyl ethers comprising alkyl or perfluoro(alkyl) chains with the alkyl group ranges from C₁₂ to C₂₄ and at least 6 fluorine atoms per perfluoroalkyl chain; C₁₂ to C₂₄ alpha-olefins such as, for example, octadecene; para-alkylstyrenes with an alkyl group comprising from 12 to 24 carbon atoms, and mixtures thereof.

As illustrations of such polymers that may be used as disclosed herein, non-limiting mention may be made of copolymers of saturated linear C₁₂ to C₃₀ alkyl (meth)acrylates forming the crystallizable portion A, and of linear C₄ to C₁₀ or branched or cyclic and/or unsaturated C₄ to C₃₀ alkyl(meth)acrylates constituting the amorphous portion B.

Among the copolymers of vinyl esters comprising linear and saturated C₁₂ to C₃₀ alkyl groups constituting the crystallizable portion A, and of vinyl esters comprising linear C₄ to C₁₀ or branched or cyclic and/or unsaturated C₄ to C₃₀ alkyl groups constituting the amorphous portion B, non-limiting mention may be made, for example, of copolymers of vinyl acetate and of vinyl stearate or of allyl stearate, such as the copolymer of allyl stearate and of vinyl acetate sold under the name “Mexomère PQ®” by the company Chimex.

When the polymers result from a polycondensation, the hydrocarbon-based and/or fluoro crystallizable chains as defined above are borne by a monomer that may be a diacid, a diol, a diamine or a diisocyanate.

b) Block Copolymers

Block copolymers comprise at least two types of blocks of different chemical nature, at least one of which is crystallizable and constitutes the portion A. In the case of block copolymers, at least one of the amorphous blocks B is soluble in the medium.

Non-limiting examples that may be mentioned include:
block copolymers of olefins or of cycloolefins comprising a crystallizable chain, for instance those derived from the block polymerization of:
   cyclobutene, cyclohexene, cyclooctene, norbornene (i.e. bicyclo[2,2,1]hept-2-ene), 5-methylnorbornene, 5-ethylnorbornene, 5,6-dimethylnorbornene, 5,5,6-trimethylnorbornene, 5-ethylidenenorbornene, 5-phenylnorbonene, 5-benzylnorbornene, 5-vinylnorbornene, 1,4,5,8-dimethano-1,2,3,4,4a,5,8a-octahydronaphthalene, dicyclopentadiene, and mixtures thereof,
   with ethylene, propylene, 1-butene, 3-methyl-1-butene, 1-hexene, 4-methyl-1-pentene, 1-octene, 1-decene, 1-eicosene, and mixtures thereof,
the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),
the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993), and “Polymer aggregates with crystalline cores: the system poly(ethylene)-poly(ethylene-propylene)” by P. Richter et al., Macromolecules, 30, 1053-1068 (1997), and
the poly(ethylene)-b-poly(ethylethylene) block copolymers cited in the general article “Crystallization in block copolymers” by I. W. Hamley, Advances in Polymer Science, Vol. 148, 113-137 (1999).

These polymers may have a single crystallizable block or a repetition of crystallizable blocks, which may be of identical or different chemical nature.

c) Copolymers Comprisnq Crystallizable End Blocks

Non-limiting examples that may be mentioned in this category include:
polycondensates of polyamide type resulting from the condensation between at least one acid chosen from dicarboxylic acids comprising at least 32 carbon atoms such as dimeric fatty acids, and alkylenediamines, for instance ethylenediamine, in which the polyamide polymer comprises at least one carboxylic acid end group esterified or amidated with at least one monoalcohol or a monoamine comprising from 12 to 30 linear and saturated carbon atoms, for instance, copolymers of ethylenediamine/stearyl dilinoleate, such as the product sold under the name “Uniclear 100 VG®” by the company Arizona Chemical; and
lipophilic polyester polycondensates, the ends of which are esterified with a crystallizable acid or alcohol comprising a saturated linear C₁₂ to C₃₀ carbon-based chain, such as 12-polyhydroxystearic acid, at least one of the ends of which is esterified with stearic acid, such as “Solsperse 21000®” sold by the company Avecia.

As further illustrations of the copolymers that may be used according to the present disclosure, non-limiting mention may be made, for example, of ethylene/vinyl acetate copolymers, ethylene/maleic anhydride copolymers, hydrogenated butadiene/isoprene block copolymers and ethylene/maleic anhydride/vinyl acetate terpolymers.

The at least one polymer that is soluble in the non-aqueous solvent medium and that has at least one crystallizable portion, can be present in the compositions as disclosed herein in an amount ranging from 0.01% to 30%, for instance from 0.1% to 20%, such as from 1% to 10% by weight, relative to the total weight of the composition.

Non-Aqueous Solvent Medium

The compositions according to the present disclosure comprise a non-aqueous solvent medium.

This medium is capable of forming a continuous phase and comprises, as its name indicates, at least one non-aqueous solvent that is generally a volatile compound, which is insoluble in water and liquid at room temperature and atmospheric pressure.

For the purposes of the present disclosure, the term “volatile compound” means any compound or non-aqueous medium capable of evaporating on contact with the skin or keratin fibers in less than 1 hour, at room temperature and atmospheric pressure. The volatile compound is a volatile cosmetic compound, which is liquid at room temperature, for example having a non-zero vapour pressure, at room temperature and atmospheric pressure, for instance having a vapour pressure ranging from 0.13 Pa to 40,000 Pa (10⁻³ to 300 mmHg), such as ranging from 1.3 Pa to 13,000 Pa (0.01 to 100 mmHg) and from 1.3 Pa to 1,300 Pa (0.01 to 10 mmHg).

In contrast, as used herein, the term “non-volatile compound” means a compound that remains on the skin or keratin fiber at room temperature and atmospheric pressure at least for several hours and that, for example, has a vapour pressure of less than 10⁻³ mmHg (0.13 Pa).

The at least one water-insoluble volatile compound that is liquid at room temperature can be present in an amount ranging from 5% to 55%, for instance from 10% to 50%, such as from 20% to 45% by weight, relative to the total weight of the composition.

For example, the at least one water-insoluble volatile compound that is liquid at room temperature may be a cosmetically acceptable organic solvent or oil. As used herein, the term “cosmetically acceptable” means a compound whose use is compatible with application to keratin fibers and to the skin.

Needless to say, the non-aqueous solvent medium of the compositions according to the present disclosure may comprise a mixture of such compounds.

The volatile oils may comprise hydrocarbon-based oils, silicone oils, fluoro oils and mixtures thereof.

As used hererin, the term “hydrocarbon-based oil” means an oil mainly comprising hydrogen and carbon atoms and possibly oxygen, nitrogen, sulfur and phosphorus atoms. The volatile hydrocarbon-based oils may be chosen from hydrocarbon-based oils comprising from 8 to 16 carbon atoms, and for example C₈-C₁₆ branched alkanes, for instance C₈-C₁₆ isoalkanes of petroleum origin (also known as isoparaffins), for instance isododecane (also known as 2,2,4,4,6-pentamethylheptane), isodecane, isohexadecane and, for example, the oils sold under the trade names “Isopars®” or “Permethyl®”, branched C₈-C₁₆ esters and isohexyl neopentanoate, and mixtures thereof. Other volatile hydrocarbon-based oils, for instance petroleum distillates, such as those sold under the name “Shell Solt®” by the company Shell, may also be used.

Volatile silicones may also be used as volatile oils, for instance volatile linear or cyclic silicone oils, such as those with a viscosity≦6 centistokes (6×10⁻⁶ m²/s) and for instance, comprising from 2 to 10 silicon atoms, these silicones optionally comprising alkyl or alkoxy groups comprising from 1 to 22 carbon atoms. Among the volatile silicone oils that may be used as disclosed herein, non-limiting mention may be made for example, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile organic solvents, for example, fluorinated volatile organic solvents, such as nonafluoromethoxybutane or perfluoromethylcyclopentane, may also be used.

In one embodiment of the compositions according to the present disclosure, for example, the water-insoluble volatile compound that is liquid at room temperature is chosen from volatile hydrocarbon-based oils comprising from 8 to 16 carbon atoms, and mixtures thereof.

The non-aqueous solvent medium may also comprise at least one water-insoluble non-volatile compound that is liquid at room temperature, such as at least one non-volatile oil, which may be chosen from, for instance, non-volatile hydrocarbon-based oils, silicone oils, and fluoro oils.

Among the non-volatile hydrocarbon-based oils that may used as disclosed herein, non-limiting mention may be made of those including, for example:
hydrocarbon-based oils of plant origin, such as triglycerides comprising fatty acid esters of glycerol, the fatty acids of which may have chain lengths ranging from C₄ to C₂₄, these chains possibly being linear or branched, and saturated or unsaturated; for instance, these oils may comprise wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil and musk rose oil; or caprylic/capric acid triglycerides, for instance those sold by the company Stearineries Dubois or those sold under the names “Miglyol 810®” “812®” and “818®” by the company Dynamit Nobel,
synthetic ethers comprising from 10 to 40 carbon atoms;
linear and branched hydrocarbons of mineral or synthetic origin, such as petroleum jelly, polydecenes, hydrogenated polyisobutene such as parleam, and squalane, and mixtures thereof;
synthetic esters, for instance oils of formula R₁COOR₂ wherein R₁ is chosen from linear and branched fatty acid residues comprising from 1 to 40 carbon atoms and R₂ is chosen from hydrocarbon-based chains, for example, branched chains, comprising from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoate, hexyl laurate, diisopropyl adipate, isononyl isononanoate, 2-ethylhexylpalmitate, isostearyl isostearate, and alcohol or polyalcohol octanoates, decanoates or ricinoleates, for instance propylene glycol dioctanoate; hydroxylated esters, for instance isostearyl lactate or diisostearyl maleate; and pentaerythritol esters;
fatty alcohols that are liquid at room temperature, with a branched and/or unsaturated carbon-based chain conmprising from 12 to 26 carbon atoms, for instance octyldodecanol, isostearyl alcohol, oleyl alcohol, 2-hexyldecanol, 2-butyloctanol and 2-undecylpentadecanol;
higher fatty acids such as oleic acid, linoleic acid and linolenic acid; and mixtures thereof.

Further non-limiting examples of non-volatile silicone oils that may be used in compositions according to the present disclosure include non-volatile polydimethylsiloxanes (PDMSs), polydimethylsiloxanes comprising alkyl or alkoxy groups, which are pendent and/or at the end of a silicone chain, these groups each comprising from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

Among the fluoro oils that may be used in the compositions of the present disclosure, include, for example, fluorosilicone oils, fluoro polyethers and fluorosilicones as described in document EP-A-847 752.

The at least one water-insoluble non-volatile compound that is liquid at room temperature may be present in an amount ranging from 0.01% to 30% by weight, for instance from 0.1% to 25% by weight, relative to the total weight of the composition.

Water and/or Water-Soluble Solvent

The compositions according to the present disclosure comprise water and/or at least one water-soluble solvent.

As disclosed herein, the term “water-soluble solvent” means a compound that is liquid at room temperature and miscible with water (water miscibility of greater than 50% by weight at 25° C. and atmospheric pressure).

In one embodiment of the present disclosure, the at least one water-soluble solvent used in the compositions as disclosed herein is volatile.

Among the water-soluble solvents that may be used in the compositions as disclosed herein, non-limiting mention may be made of, for instance, lower monoalcohols comprising from 1 to 5 carbon atoms, such as ethanol and isopropanol, glycols comprising from 2 to 8 carbon atoms, such as ethylene glycol, propylene glycol, 1,3-butylene glycol and dipropylene glycol, C₃ and C₄ ketones and C₂-C₄ aldehydes.

The water and/or the at least one water-soluble solvent may be introduced as such into the formulation according to the present disclosure, or may be incorporated therein by means of at least one ingredient comprising the composition. Thus, water may be introduced into the composition, for example, via the introduction of latex or pseudolatex, i.e. an aqueous dispersion of polymer particles.

The presence of water and/or of at least one water-soluble solvent in the compositions can make it possible to increase the adhesion of the composition to the eyelashes. For example, the larger the amount of the at least one non-aqueous solvent, the more slippery the application onto the eyelashes, on account of the mainly “oily” nature of the composition. Thus the partial replacement of the at least one non-aqueous solvent with water and/or at least one water-soluble solvent makes it possible to reduce this effect and to increase the adhesion to the eyelashes. The makeup result obtained is then thicker.

With respect to waterproof mascaras, the amount of water and/or at least one water-soluble solvent present in the compositions is, however, generally less than or equal to 20%, and for instance, is greater than or equal to 0.5% by weight, relative to the total weight of the composition.

The amount of water and/or at least one water-soluble solvent in the compositions of the present disclosure may range, for example, from 1% to 18%, such as from 2% to 15% by weight, relative to the total weight of the composition.

Waxes

The compositions according to the present disclosure comprise at least one wax or a mixture of waxes in an amount greater than 3% by weight, relative to the total weight of the composition.

The waxes that may be used in the context of the present disclosure are generally lipophilic compounds that are solid at room temperature (25° C.), with a solid/liquid reversible change of state, having a melting point of greater than or equal to 30° C., which may be up to 200° C., for instance up to 120° C.

By bringing the at least one wax to the liquid form (melting), it is possible to make it miscible with oils and to form a microscopically uniform mixture, but on cooling the mixture to room temperature, recrystallization of the at least one wax in the oils of the mixture is obtained.

For example, the waxes that may be used as dislosed herein can have a melting point of greater than or equal to 45° C., for instance, greater than or equal to 55° C.

For the purposes of the present disclosure, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name “MDSC 2920” by the company TA Instruments.

The measuring protocol is as follows: A sample of 5 mg of product placed in a crucible is subjected to a first temperature rise ranging from −20° C. to 100° C., at a heating rate of 10° C./minute, it is then cooled from 10° C. to −20° C. at a cooling rate of 10° C./minute and is then subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the at least one wax is the temperature value corresponding to the top of the peak of the curve representing the variation in the difference in absorbed power as a function of the temperature.

The waxes that may be used in the compositions according to the present disclosure are chosen from waxes that are solid at room temperature, of animal, plant, mineral or synthetic origin, and mixtures thereof.

The waxes that may be used in the compositions according to the present disclosure generally have a hardness ranging from 0.01 MPa to 15 MPa, for instance greater than 0.05 MPa, such as greater than 0.1 MPa.

The hardness is determined by measuring the compression force, measured at 20° C. using a texturometer sold under the name “TA-XT2i®” by the company Rheo, equipped with a stainless-steel cylindrical spindle 2 mm in diameter, by measuring the change in force (compression force or stretching force) (F) as a function of time, during the following operation:

The spindle is displaced at a speed of 0.1 mm/s and then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.1 mm/s. During the relaxation time, the force (compression force) decreases greatly until it becomes zero, and then, during the withdrawal of the spindle, the force (stretching force) becomes negative and then rises again towards the value 0. The hardness corresponds to the maximum compression force measured between the surface of the spindle and the wax at the moment they come into contact. The value of this force is expressed in MPa.

To measure the hardness, the wax is melted at a temperature equal to the melting point of the wax+20° C. The molten wax is poured into a container 30 mm in diameter and 20 mm deep. The wax is recrystallized at room temperature (25° C.) for 24 hours and is then stored for at least 1 hour at 20° C., before performing the hardness measurement.

As illustrations of waxes that are suitable for use as disclosed herein, non-limiting mention may be made of, for example, hydrocarbon-based waxes, for instance beeswax, lanolin wax, Chinese insect waxes, sumach wax, paraffins, certain polyethylene waxes and waxy copolymers, and also esters thereof.

Non-limiting mention may also be made of waxes obtained by catalytic hydrogenation of animal and plant oils comprising linear or branched C₈-C₃₂ fatty chains. Further among these waxes that may be used, non-limiting mention may be made of isomerized jojoba oil such as the trans-isomerized partially hydrogenated jojoba oil manufactured or sold by the company Desert Whale under the commercial reference “Iso-Jojoba-50®”, hydrogenated sunflower oil, hydrogenated castor oil, hydrogenated coconut oil, hydrogenated lanolin oil and bis(1,1,1-trimethylolpropane)tetrastearate sold under the name “Hest 2T-4S®” by the company Heterene.

Non-limiting mention may also be made of silicone waxes and fluoro waxes.

The waxes obtained by hydrogenation of castor oil esterified with cetyl alcohol, sold under the names “Phytowax ricin 16L64®” and “22L73® by the company Sophim may also be used. Such waxes are described in French Patent Application FR-A-2,792,190.

According to one embodiment of the present disclosure, the compositions as disclosed herein may comprise at least one “tacky” wax, i.e. a wax with a tack of greater than or equal to 0.7 N.s, and a hardness of less than or equal to 3.5 MPa.

Using a tacky wax can, for example, make it possible to obtain a cosmetic composition that can for instance, apply easily to keratin fibers, attach well to the keratin fibers, and lead to the formation of a smooth, uniform and thickening makeup result.

The at least one tacky wax that may be used as disclosed herein can have a tack ranging from 0.7 N.s to 30 N.s, for instance, greater than or equal to 1 N.s, such as ranging from 1 N.s to 20 N.s, for instance, greater than or equal to 2 N.s, such as ranging from 2 N.s to 10 N.s and ranging from 2 N.s to 5 N.s.

The tack of the wax is determined by measuring the change in force (compression force or stretching force) as a function of time, at 20° C., using the texturometer sold under the name “TA-XT2i®” by the company Rheo, equipped with a conical acrylic polymer spindle forming an angle of 45°. The measuring protocol is as follows:

The wax is melted at a temperature equal to the melting point of the wax+10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s. During the relaxation time, the force (compression force) decreases until it becomes zero, and then, during the withdrawal of the spindle, the force (stretching force) becomes negative and then rises again to the value zero. The tack corresponds to the integral of the curve of the force as a function of time for the part of the curve corresponding to negative values of the force (stretching force). The tack value is expressed in N.s.

The at least one tacky wax that may be used can have a hardness of less than or equal to 3.5 MPa, for instance, ranging from 0.01 MPa to 3.5 MPa, such as ranging from 0.05 MPa to 3 MPa, and ranging from 0.1 MPa to 2.5 MPa.

The hardness is measured according to the protocol described herein.

Non-limiting examples of tacky waxes that may be used as disclosed herein include C₂₀-C₄₀ alkyl(hydroxystearyloxy)stearates, alone or as a mixture, for instance C₂₀-C₄₀ alkyl 12-(12′-hydroxystearyloxy)stearates, of formula (II): 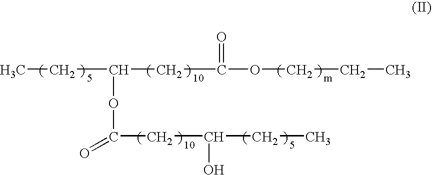
wherein m is an integer ranging from 18 to 38, or a mixture of compounds of formula (II).

Such waxes are sold, for instance, under the names “Kester Wax K 82 P®” and “Kester Wax K 80 P®” by the company Koster Keunen.

The waxes described above can have a starting melting point of less than 45° C.

According to one embodiment, the compositions disclosed herein comprise at least one wax with a high starting melting point, i.e. greater than or equal to 45° C., for instance, greater than or equal to 50° C., or even a very high starting melting point, i.e. greater than or equal to 55° C., such as greater than or equal to 60° C. The starting melting point of the wax may be measured using a differential scanning calorimeter (DSC), for example the calorimeter sold under the name “MDSC2920®” by the company TA Instruments.

The measuring protocol is the same as that described for measuring the melting point.

The starting melting point, denoted hereinbelow by the abbreviation “mpₛₜₐᵣₜ,” of the compound corresponds to the temperature measured when 5% of the heat of fusion is consumed.

Among waxes with a starting melting point that is high but less than 50° C., non-limiting mention may be made of, for example, montan wax (mpₛₜₐᵣₜ=47.9° C.), ozokerite (mpₛₜₐᵣₜ=46.3° C.) and the wax obtained by catalytic hydrogenation of olive oil esterified with stearyl alcohol, sold under the name “Phytowax Olive 18L57®” by the company Sophim (mpₛₜₐᵣₜ=47.4° C.).

Among waxes with a starting melting point of greater than or equal to 50° C. and less than 55° C., non-limiting mention may be made for instance, of rice bran wax (mpₛₜₐᵣₜ=51.4° C.), candelilla wax (mpₛₜₐᵣₜ=50° C.) and ouricurry wax (mpₛₜₐᵣₜ=51.4° C.).

Among waxes with a very high starting melting point that may be as disclosed herein, non-limiting mention may be made of, for example, carnauba wax (mpₛₜₐᵣₜ=63.5° C.), the waxes obtained by Fisher-Tropsch synthesis (mpₛₜₐᵣₜ=60.5° C.), certain polyethylene waxes such as, for instance, those sold under the name “Performalene 655®” by the company New Phase Technologies or “Polyethylene AC 540®” by the company Honeywell, “Polywax 2000 T-6®” by the company Petrolite (mpₛₜₐᵣₜ=125° C.) or “PED 191®” and “Epolene N-14®” by the company Eastman Kodak (mpₛₜₐᵣₜ=120° C. and 106° C., respectively) and certain monocrystalline waxes such as those sold under the names “Tisco Wax 88®” by the company Tisco or “Microwax HW®” by the company Paramelt.

Further among the waxes with a very high starting melting point, non-limiting mention may also be made of waxes obtained by catalytic hydrogenation of animal or plant oils comprising linear or branched C₈-C₃₂ fatty chains, such as hydrogenated jojoba oil (mpₛₜₐᵣₜ=63.2° C.) and bis(1,1,1-trimethylolpropane)tetrabehenate sold under the name “Hest 2T4B®” by the company Heterene (mpₛₜₐᵣₜ=61.8° C.).

The wax with a high, or very high, starting melting point may be present in the compositions as disclosed herein in large amount. For example, in one embodiment of the present disclosure, the at least one wax with a high or very high starting melting point is present in the composition in an amount greater than or equal to 20% by weight, relative to the total weight of the composition.

According to the present disclosure, it is also possible to use waxes supplied in the form of small particles ranging from 0.5 to 30 micrometers, for instance, from 1 to 20 micrometers, such as from 5 to 10 micrometers in size, which will be referred to in the present disclosure as “microwaxes.” For the purposes of distinction, the waxes used according to the present disclosure in the form of larger fragments are referred to as “conventional waxes.”

Among the microwaxes that may be used in the compositions as disclosed herein, non-limiting mention may be made of carnauba microwaxes, such as the product sold under the name “MicroCare 350®” by the company Micro Powders, synthetic microwaxes, such as that product sold under the name “MicroEase 114S®” by the company Micro Powders, microwaxes comprising a mixture of carnauba wax and polyethylene wax, such as the products sold under the names “Micro Care 300®” and “310®” by the company Micro Powders, microwaxes comprising a mixture of carnauba wax and of synthetic wax, such as the product sold under the name “Micro Care 325®” by the company Micro Powders, polyethylene microwaxes, such as the products sold under the names “Micropoly 200®”, “220®”, “220L®” and “250S®” by the company Micro Powders, and polytetrafluoroethylene micropowders such as the products sold under the names “Microslip 519®” and “519 L®” by the company Micro Powders.

Among the microwaxes mentioned above, some of them, for instance carnauba microwax, the synthetic microwax “MicroEase 114S®” or the microwax comprising a mixture of carnauba wax and of synthetic wax “MicroCare 325®”, have a starting melting point of greater than or equal to 45° C.

In the compositions as disclosed herein, it is possible to use a mixture of waxes, for example, to use at least one conventional wax, such as, for instance, at least one tacky wax and/or at least one wax with a starting melting point of greater than or equal to 45° C., and at least one microwax.

The compositions according to the present disclosure can comprise a total amount of wax ranging from 10% to 70% by weight, relative to the total weight of the composition. For example, the total amount of wax may be present in an amount ranging from 15% to 65%, for instance, from 20% to 60%, such as from 25% to 55% by weight, relative to the total weight of the composition.

The at least one wax or mixture of waxes is present in the compositions according to the present disclosure in the form of a dispersion of particles in the non-aqueous solvent medium.

The wax particles may have varied shapes. In one embodiment of the present disclosure, the wax particles are spherical.

Microscopic observation of a sample of a composition as disclosed herein, at room temperature, shows a good dispersion of the wax particles in the medium, with little or no aggregation of these particles, for example, a distribution of the particles that is substantially identical in all directions.

Film-Forming Polymer

According to another embodiment of the present disclosure, the compositions as disclosed herein may comprise at least one film-forming polymer.

As disclosed herein, the term “film-forming polymer” means a polymer capable, by itself or in the presence of an auxiliary film-forming agent, of forming a continuous film that adheres to a support, such as keratin materials.

Film-forming polymers according to the present disclosure, include liposoluble polymers comprising less than 30% by weight of a crystallizable portion as disclosed herein, or for example, comprising no crystallizable portion at all.

Among the film-forming polymers that may be used in the compositions of the present disclosure, non-limiting mention may be made of synthetic polymers, of free-radical type or of polycondensate type, and polymers of natural origin, and mixtures thereof.

Non-limiting examples of liposoluble polymers that may be used as disclosed herein include copolymers of vinyl esters wherein the vinyl group is directly linked to the oxygen atom of the ester group and vinyl esters comprising a saturated, linear or branched hydrocarbon-based radical of 1 to 24 carbon atoms, linked to the carbonyl of the ester group, and of at least one other monomer, which may be chosen from vinyl esters different from the vinyl ester already present, alkyl vinyl ethers the alkyl group of which comprises from 2 to 24 carbon atoms, and allylic and methallylic esters comprising a saturated, linear or branched hydrocarbon-based radical of 1 to 24 carbon atoms, linked to the carbonyl of the ester group. These copolymers may be crosslinked using crosslinking agents that may be of the vinylic type, of the allylic and methallylic type, such as tetraallyloxyethane, divinylbenzene, divinyl octanedioate, divinyl dodecanedioate and divinyl octadecanedioate.

Further non-limiting examples of these copolymers that may be used as disclosed herein include the copolymers: vinyl acetate/vinyl laurate, vinyl propionate/allyl laurate, vinyl propionate/vinyl laurate, and allyl 2,2-dimethylpentanoate/vinyl laurate.

Among additional liposoluble film-forming polymers that may also be used as disclosed herein, non-limiting mention may be made of liposoluble homopolymers, for instance, those resulting from the homopolymerization of vinyl esters comprising from 9 to 22 carbon atoms or of alkyl acrylates or methacrylates, the alkyl radicals comprising from 2 to 24 carbon atoms.

Still additional non-limiting examples of liposoluble homopolymers that may be used include those chosen from polyvinyl laurate and polylauryl(meth)acrylates, these poly(meth)acrylates possibly being crosslinked using ethylene glycol dimethacrylate or tetraethylene glycol dimethacrylate.

The liposoluble homopolymers and copolymers defined above are known and described, for example, in French Patent Application FR-A-2,232,303; they may have a weight-average molecular weight ranging from 2,000 to 500,000, for instance, from 4000 to 200,000.

Among liposoluble film-forming polymers that may be used as disclosed herein, non-limiting mention may also be made of polyalkylenes, for example C₂ to C₂₀ alkene copolymers, for instance polybutene, alkylcelluloses with a linear or branched, saturated or unsaturated C₁ to C₈ alkyl radical, for instance ethylcellulose and propylcellulose, vinylpyrrolidone (VP) copolymers, such as copolymers of vinylpyrrolidone and of a C₂ to C₄₀, for example, C₃ to C₂₀, alkene. As examples of VP copolymers that may be used in the invention, non-limiting mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene and VP/acrylic acid/lauryl methacrylate copolymers.

The at least one film-forming polymer may also be present in the composition in the form of particles dispersed in an aqueous phase or in a non-aqueous solvent phase, which is generally known as a latex or a pseudolatex. The techniques for preparing these dispersions are known to those skilled in the art.

Non-limiting examples of aqueous dispersions of at least one film-forming polymer that may be used include the acrylic dispersions sold under the names “Neocryl XK-90®,” “Neocryl A-1070®,” “Neocryl A-1090®,” “Neocryl BT-62®,” “Neocryl A-1079®” and “Neocryl A-523®” by the company Avecia-Neoresins, “Dow Latex 432®” by the company Dow Chemical, “Daitosol 5000 AD®” by the company Daito Kasey Kogyo; or the aqueous dispersions of polyurethane sold under the names “Neorez R-981®” and “Neorez R-974®” by the company Avecia-Neoresins, “Avalure UR-405®,” “Avalure UR-410®,” “Avalure UR-425®,” “Avalure UR-450®,” “Sancure 875®,” “Sancure 861®,” “Sancure 878®” and Sancure 2060®” by the company Goodrich, “Impranil 85®” by the company Bayer, “Aquamere H-1511®” by the company Hydromer; the sulfopolyesters sold under the brand name “Eastman AQ®” by the company Eastman Chemical Products, vinylic dispersions, for instance “Mexomère PAM” and also acrylic dispersions in isododecane, for instance “Mexomère PAP” by the company CHIMEX.

The at least one film-forming polymer may be present in the composition as disclosed herein in a solids content ranging from 0.1% to 30% by weight, for instance, from 0.5% to 25% by weight, such as from 1% to 20% by weight relative to the total weight of the composition.

The compositions according to the present disclosure may also comprise a plasticizer to promote the formation of a film with the film-forming polymer. Such a plasticizer may be chosen from any compound known to those skilled in the art as being capable of satisfying the desired function.

Dyestuff

The compositions according to the present disclosure may also comprise at least one dyestuff, for instance pulverulent dyes, liposoluble dyes and water-soluble dyes.

The at least one pulverulent dyestuff can be chosen from pigments and nacres.

The at least one pigment can be white or colored, mineral and/or organic, and coated or uncoated. Among the mineral pigments that may be used as disclosed herein, non-limiting mention may be made of titanium dioxide, optionally surface-treated, zirconium oxide, zinc oxide, cerium oxide, iron oxide, chromium oxide, manganese violet, ultramarine blue, chromium hydrate and ferric blue. Among the organic pigments that may be used as disclosed herein, non-limiting mention may be made of carbon black, pigments of D & C type, and lakes based on cochineal carmine or on barium, strontium, calcium or aluminium.

The at least one nacre can be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the abovementioned type, and also nacreous pigments based on bismuth oxychloride.

The at least one liposoluble dye can be chosen from, for example, Sudan Red, D&C Red 17, D&C Green 6, β-carotene, soybean oil, Sudan Brown, D&C Yellow 11, D&C Violet 2, D&C Orange 5, quinoline yellow and annatto.

These dyestuffs may be present in an amount ranging from 0.01% to 30% by weight, relative to the total weight of the composition.

Fillers

The composition according to the present disclosure may also comprise at least one filler.

The at least one filler may be chosen from those that are known to persons skilled in the art and commonly used in cosmetic compositions. The at least one filler may be chosen from mineral and organic materials, and lamellar and spherical fibers. Non-limiting mention may be made of talc, mica, silica, kaolin, polyamide powders, for instance the Nylon® sold under the trade name “Orgasol®” by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name “Expancel®” by the company Nobel Industrie, acrylic powders, such as those sold under the name “Polytrap®” by the company Dow Corning, polymethyl methacrylate particles and silicone resin microbeads for example “Tospearls®” from Toshiba, precipitated calcium carbonate, magnesium carbonate and magnesium hydrocarbonate, hydroxyapatite, hollow silica microspheres such as “Silica Beads®” from Maprecos, glass or ceramic microcapsules, metal soaps derived from organic carboxylic acids comprising from 8 to 22 carbon atoms, for instance, from 12 to 18 carbon atoms, for example zinc, magnesium or lithium stearate, zinc laurate and magnesium myristate.

The at least one filler may be present in a total amount of fillers ranging from 0.1% to 25%, such as from 1% to 20% by weight, relative to the total weight of the composition.

The compositions of the present disclosure may also comprise at least one cosmetically acceptable additive chosen for example, from those conventionally used in cosmetics, such as antioxidants, preserving agents, fragrances, neutralizers, plasticizers, thickeners, gelling agents, fibers, cosmetic active agents, and mixtures thereof.

The at least one gelling agent that may be used in the compositions according to the present disclosure may be lipophilic, and may be organic or mineral, and polymeric or molecular.

Non-limiting examples of mineral lipophilic gelling agents that may be used as disclosed herein include optionally modified clays, for instance hectorites modified with a C₁₀ to C₂₂ fatty acid ammonium chloride, for instance hectorite modified with distearyldimethylammonium chloride, for instance the product sold under the name “Bentone 38V®” by the company Elementis.

Non-limiting mention may also be made of fumed silica optionally subjected to a hydrophobic surface treatment, the particle size of which is less than 1 μm. For example, it is possible to chemically modify the surface of the silica, by chemical reaction generating a reduced number of silanol groups present at the surface of the silica. It is for instance, possible to substitute silanol groups with hydrophobic groups: a hydrophobic silica is then obtained. The hydrophobic groups that may be used as disclosed herein, include:
trimethylsiloxyl groups, which are obtained for example, by treating fumed silica in the presence of hexamethyldisilazane. Silicas thus treated are known as “silica silylate” according to the CTFA (6th edition, 1995). They are sold, for example, under the names “Aerosil R812®” by the company Degussa, and “Cab-O-Sil TS-530®” by the company Cabot;
dimethylsilyloxyl or polydimethylsiloxane groups, which are obtained for example, by treating fumed silica in the presence of polydimethylsiloxane or dimethyldichlorosilane. Silicas thus treated are known as “silica dimethyl silylate” according to the CTFA (6th edition, 1995). They are sold, for example, under the names “Aerosil R972®” and “Aerosil R974®” by the company Degussa, and “Cab-O-Sil TS-610®” and “Cab-O-Sil TS-720®” by the company Cabot.

The hydrophobic fumed silica for example, may have a particle size that may be nanometric to micrometric, for example ranging from about 5 to 200 nm.

The polymeric organic lipophilic gelling agents are, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SR DMF 10®, SR-DC556®, SR 5CYC gel®, SR DMF 10 gel® and SR DC 556 gel® from Grant Industries and SF 1204® and JK 113® from General Electric; ethylcellulose, for instance those sold under the name Ethocel by Dow Chemical and galactomannans comprising from one to six, such as from two to four, hydroxyl groups per saccharide, substituted with a saturated or unsaturated alkyl chain, for instance guar gum alkylated with C₁ to C₆, such as C₁ to C₃, alkyl chains, and mixtures thereof. Block copolymers of “diblock” or “triblock” type, of the polystyrene/polyisoprene or polystyrene/polybutadiene type such as the products sold under the name “Luvitol HSB®” by the company BASF, of the polystyrene/copoly(ethylene-propylene) type such as the products sold under the name “Kraton®” by the company Shell Chemical Co., or of the polystyrene/copoly(ethylene-butylene) type may also be used.

Among the gelling agents that may be used in the compositions according to the present disclosure, non-limiting mention may also be made of fatty acid esters of dextrin, such as dextrin palmitates, such as the products sold under the name “Rheopearl TL®” or “Rheopearl KL®” by the company Chiba Flour.

The composition according to the present disclosure may also comprise fibers to allow an improvement in the lengthening effect.

The term “fiber” should be understood as meaning an object of length L and diameter D such that L is much greater than D, D being the diameter of the circle in which the cross section of the fiber is inscribed. For example, the ratio L/D (or shape factor) of the fiber ranges from 3.5 to 2,500, for instance, from 5 to 500, such as from 5 to 150.

The fibers that may be used in the composition of the invention are chosen from mineral, organic, synthetic, and natural fibers. They may be short or long, individual or organized, for example braided, and hollow or solid. They may have any shape, for instance, may have a circular or polygonal such as square, hexagonal or octagonal cross sections, depending on the intended application of the fibers. Further, their ends may be blunt and/or polished to prevent injury.

For example, the fibers can have a length ranging from 1 μm to 10 mm, for instance from 0.1 mm to 5 mm, such as from 1 mm to 3.5 mm. The cross section of the fibers may be within a circle of diameter ranging from 2 nm to 500 μm, for instance ranging from 100 nm to 100 μm, such as from 1 μm to 50 μm. The weight or yarn count of the fibers is often given in denier or decitex, and is the weight in grams per 9 km of yarn. For example, the fibers according to the present disclosure may have a yarn count ranging from 0.15 to 30 denier, such as from 0.18 to 18 denier.

The fibers can be those used in the manufacture of textiles, and in particular silk fiber, cotton fiber, wool fiber, flax fiber, cellulose fiber extracted for example, from wood, from plants or from algae, rayon fiber, polyamide (Nylon®) fiber, viscose fiber, acetate fiber, such as rayon acetate fiber, poly(p-phenyleneterephthalamide) or aramide fiber, for instance Kevlar® fiber, acrylic polymer fiber, for example polymethyl methacrylate fiber or poly(2-hydroxyethyl methacrylate) fiber, polyolefin fiber such as polyethylene or polypropylene fiber, glass fiber, silica fiber, carbon fiber, for instance in graphite form, polytetrafluoroethylene (such as Teflon®) fiber, insoluble collagen fiber, polyester fiber, polyvinyl chloride fiber or polyvinylidene chloride fiber, polyvinyl alcohol fiber, polyacrylonitrile fiber, chitosan fiber, polyurethane fiber, polyethylene phthalate fiber, and fibers formed from a mixture of polymers such as those mentioned above, for instance polyamide/polyester fibers.

The fibers used in surgery may also be used, for instance the resorbable synthetic fibers prepared from glycolic acid and caprolactone (Monocryl® from Johnson & Johnson); resorbable synthetic fibers of the type which is a copolymer of lactic acid and of glycolic acid (Vicryl® from Johnson & Johnson); polyterephthalic ester fibers (Ethibond® from Johnson & Johnson) and stainless steel threads (Acier® from Johnson & Johnson).

Moreover, the fibers may be treated or untreated at the surface, and coated or uncoated. Among coated fibers that may be used as disclosed herein, non-limiting mention may be made of polyamide fibers coated with copper sulphide to give an anti-static effect (for example R-STAT® from Rhodia) or another polymer enabling a particular organization of the fibers such as via a specific surface treatment or surface treatment including color/hologram effects for example, Lurex® fiber from Sildorex.

In one embodiment of the present disclosure, fibers of synthetic origin, for instance organic fibers, such as those used in surgery, are used. In yet another embodiment, water-insoluble fibers are used.

Further among the fibers that may be used in the composition according to the present disclosure, non-limiting mention may be made of, for example, polyamide fibers, cellulose fibers, poly(p-phenyleneterephthalamide) fibers or polyethylene fibers. Their length L may range from 0.1 mm to 5 mm, for instance, from 0.25 mm to 1.6 mm, and their mean diameter may range from 1 μm to 50 μm. For example, the polyamide fibers sold by Etablissements P. Bonte under the name “Polyamide 0.9 Dtex 3 mm®,” having a mean diameter of 6 μm, a yarn count of about 0.9 dtex and a length ranging from 0.3 mm to 5 mm, may be used. Cellulose or rayon fibers with a mean diameter of 50 μm and a length ranging from 0.5 mm to 6 mm may also be used, for instance those sold under the name “Natural rayon flock fiber RC1BE-N003-M04®” by the company Claremont Flock. Polyethylene fibers, for instance those sold under the name “Shurt Stuff 13 099 F®” by the company Mini Fibers, may also be used.

The composition according to the present disclosure may also comprise “rigid” fibers, as opposed to the fibers described above, which are not rigid fibers.

The rigid fibers, which are initially substantially straight, when placed in a dispersing medium, do not undergo a substantial change in shape, which is reflected by the angular condition defined below, reflecting a shape that may be described as still substantially straight and linear. This angle condition reflects the stiffness of the fibers, which it is difficult to express by another parameter for objects that are as small as the rigid fibers.

The stiffness of the fibers is reflected by the following angular condition: for example, at least 50%, for instance at least 75% such as at least 90%, in numerical terms, of the fibers are such that the angle formed between the tangent to the longitudinal central axis of the fiber and the straight line connecting the end to the point on the longitudinal central axis of the fiber corresponding to half the length of the fiber is less than 15°, and the angle formed between the tangent to the longitudinal central axis of the fiber at a point half way along the fiber and the straight line connecting one of the ends to the point on the longitudinal central axis of the fiber corresponding to half the length of the fiber, is less than or equal to 150 for the same fiber length ranging from 0.8 mm to 5 mm, for instance ranging from 1 mm to 4 mm, such as ranging from 1 mm to 3 mm, or even 2 mm.

For example, the angle mentioned above is measured at the two ends of the fiber and at a point half way along the fiber; in other words, three measurements are taken and the average of the measured angles is less than or equal to 15°.

In one embodiment of the present disclosure, the tangent, at any point on the fiber, forms an angle of less than 15°.

For the purposes of the present disclosure, the angle formed by the tangent at a point on the fiber is the angle formed between the tangent to the longitudinal central axis of the fiber at the point on the fiber and the straight line connecting the end of the fiber that is closest to the point on the longitudinal central axis of the fiber corresponding to half the length of the fiber.

Generally, the rigid fibers that may be used in the composition according to the present disclosure have the same or substantially the same fiber length.

For example, when a medium that has rigid fibers dispersed in an amount of 1% by weight is observed by microscope, with an objective lens allowing a magnification of 2.5 and with full-field vision, a numerical majority of the rigid fibers, i.e. at least 50% numerically of the rigid fibers, for instance at least 75% numerically of the rigid fibers such as at least 90% numerically of the rigid fibers, must satisfy the angular condition defined above. The measurement leading to the angle value is performed for the same length of fibers, this length ranging from 0.8 mm to 5 mm, for instance from 1 to 4 mm, such as from 1 to 3 mm, and even 2 mm.

The medium in which the observation is performed is a dispersing medium that ensures good dispersion of the rigid fibers, for example water or an aqueous gel of clay or of associative polyurethane. A direct observation of the composition comprising the rigid fibers may also be performed. A sample of the prepared composition or dispersion is placed between a slide and cover slip for observation by microscope with an objective lens allowing a magnification of 2.5 and with full-field vision. Full-field vision allows the fibers to be viewed in their entirety.

The rigid fibers may be chosen from fibers of a synthetic polymer chosen from polyesters, polyurethanes, acrylic polymers, polyolefins, polyamides, such as non-aromatic polyamides, and aromatic polyimideamides.

Non-limiting examples of rigid fibers that may be used as disclosed herein include:
polyester fibers, such as those obtained by chopping yarns sold under the names Fiber 255-100-R11-242T Taille 3 mm (eight-lobed cross section)®, Fiber 265-34-R11-56T Taille 3 mm (round cross section)® and Fiber Coolmax 50-34-591 Taille 3 mm (four-lobed cross section)® by the company Dupont de Nemours;
polyamide fibers, such as those sold under the names Trilobal Nylon 0.120-1.8 DPF®; Trilobal Nylon 0.120-18 DPF®; Nylon 0.120-6 DPF by the company Cellusuede Products; or obtained by chopping yarns sold under the name Fiber Nomex Brand 430 Taille 3 mm® by the company Dupont de Nemours;
polyimideamide fibers, such as those sold under the names “Kermel®” and “Kermel Tech®” by the company RHODIA;
poly(p-phenyleneterephthalamide) (or aramide) sold especially under the name Kevlar® by the company Dupont de Nemours;
fibers with a multilayer structure comprising alternating layers of polymers chosen from polyesters, acrylic polymers and polyamides, such as those described in European Patent Nos. EP-A-6,921,217 and EP-A-686,858 and U.S. Pat. No. 5,472,798. Such fibers are sold under the names “Morphotex®” and “Teijin Tetron Morphotex®” by the company Teijin.

In one embodiment of the present disclosure, the rigid fibers that are are aromatic polyimideamide fibers.

Polyimideamide yarns or fibers that may be used for the compositions of the present disclosure are described, for example, in the document from R. Pigeon and P. Allard, Chimie Macromoléculaire Appliquée, 40/41 (1974), pages 139-158 (No. 600), or in U.S. Pat. No. 3,802,841, French Patent No. FR-A-2,079,785, and European Patent Nos. EP-A1-0 360 728 and EP-A-0 549 494, to which reference may be made.

For example, the aromatic polyimideamide fibers may be polyimideamide fibers comprising repeating units of formula: 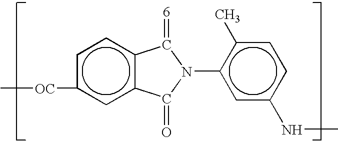
obtained by polycondensation of tolylene diisocyanate and trimellitic anhydride.

The fibers may be present in the composition according to the present disclosure in an amount ranging from 0.01% to 10% by weight, for instance, from 0.1% to 5% by weight, such as from 0.3% to 3% by weight relative to the total weight of the composition.

Among the cosmetic active agents that may be used in the compositions as disclosed herein, non-limiting mention may be made for example, of emollients, moisturizers, vitamins and screening agents, such as sunscreens.

Needless to say, a person skilled in the art will take care to select the optional additional additives and/or the amount thereof such that the beneficial properties of the compositions according to the present disclosure are not, or are not substantially, adversely affected by the envisaged addition.

Rheological Characteristics

The compositions in accordance with the present disclosure are characterized by viscoelastic behaviour.

As disclosed herein, a material is said to be viscoelastic when, under the effect of shear, it has both the characteristics of a purely elastic material, i.e. capable of storing energy, and the characteristics of a purely viscous material, i.e. capable of dissipating energy.

For example, the viscoelastic behavior of the compositions in accordance with the present disclosure may be characterized by its modulus of rigidity G, its elasticity δ and its flow threshold τ_{c}; these parameters are defined for instance, in the publication “Initiation à la rhéologie [Introduction to Rheology]”, G. Couarraze and J. L. Grossiord, 2nd edition, 1991, published by Lavoisier-Tec 1 Doc. These parameters are measured by means of measurements performed at 25° C.±0.5° C. using a Haake RheoStress 600® controlled-stress rheometer from the company ThermoRheo, equipped with a stainless-steel rotor with plate/plate geometry, the plate having a diameter of 20 mm and a gap (distance between the lower plate—known as the stator plate—on which the composition is deposited, and the upper plate—known as the rotor plate) of 0.3 mm. The two plates are striated to limit the sliding phenomena to the walls of the plates.

The dynamic measurements are performed by applying a harmonic variation of the stress. In these experiments, the magnitudes of the shear, the shear rate and the stress are low so as to remain within the limits of the linear viscoelastic domain of the material (conditions for evaluating the rheological characteristics of the composition at rest).

The linear viscoelastic domain is defined by the fact that the response of the material (i.e. the strain) is at any moment directly proportional to the value of the applied force (i.e. the stress). In this domain, the applied stresses are small and the material undergoes strains without modifying its microscopic structure. Under these conditions, the material is studied “at rest” and non-destructively.

The composition is subjected to a harmonic shear according to a stress τ(t) varying sinusoidally according to a pulse ω (ω=2πν, ν being the frequency of the applied shear). The composition thus sheared undergoes a stress τ(t) and responds according to a strain γ(t) corresponding to micro-strains for which the modulus of rigidity varies little as a function of the imposed stress.

The stress τ(t) and the strain γ(t) are defined, respectively, by the following relationships:
τ(t)=Σ₀ cos(ω·t) γ(t)=γ₀ cos(ω·t−δ)
τ₀ being the maximum amplitude of the stress and γ₀ being the maximum amplitude of the strain. δ is the dephasing angle between the stress and the strain.

The measurements are performed at a frequency of 1 Hz (ν=1 Hz).

The change in the modulus of rigidity G (corresponding to the ratio of τ₀ to γ₀) and in the elasticity δ (corresponding to the dephasing angle of the applied stress relative to the measured strain) as a function of the applied stress τ(t) are thus measured.

The strain of the composition is measured for example, for the stress region in which the variation of the modulus of rigidity G and of the elasticity δ is less than 7% (micro-strain zone), and thus the “plateau” parameters Gp and δₚ are determined. The threshold stress τ_{c}, which is the minimum force that it is necessary to apply to the composition to cause it to flow, is determined from the curve δ=f(τ) and corresponds to the value of τ for which δ(τ_{c})=1.05 δₚ.

The viscoelastic behaviour of the compositions according to the present disclosure may be characterized, for example, by a plateau modulus of rigidity Gp of less than or equal to 35,000 Pa, for instance less than or equal to 30,000 Pa, such as less than or equal to 28,000 Pa and less than or equal to 25,000 Pa, and even 20,000 Pa.

The compositions in accordance with the present disclosure may moreover have a flow threshold τ_{c} ranging from 10 Pa to 200 Pa, and for instance, ranging from 20 Pa to 100 Pa.

Preparation Process

The process for preparing the compositions according to the present disclosure depends for example on the nature of the at least one wax used. For instance, it depends on whether the at least one wax is of conventional type or of microwax type as defined above. For the conventional waxes, it furthermore depends on the starting melting point of the wax.

In one embodiment of the present disclosure, the at least one wax used is chosen from those of conventional type and have a starting melting point of less than 45° C.

In such a case, the compositions as disclosed herein may be prepared in a standard manner by heating the at least one wax until completely melted and then introducing the wax into a volatile non-aqueous solvent. The mixture thus obtained is subjected to mechanical stirring until it has cooled to room temperature. The polymer that is soluble in the non-aqueous solvent medium and that has a crystallizable portion in accordance with the present disclosure may be introduced with the volatile non-aqueous solvent, or may also be introduced subsequent thereto.

The water and/or at least one water-soluble solvent and the optional additional ingredients may be introduced into the starting materials or, optionally, during the cooling or into the finished composition.

In another embodiment of the present disclosure, the at least one wax used is of conventional type, wherein the starting melting point may be less than, equal to or greater than 45° C. In this embodiment, the keratin fiber makeup compositions are obtained by heating the at least one wax to a temperature above the melting point of the wax that has the highest melting point, until they have completely melted, followed by blending and continuous cooling to room temperature.

According to still another embodiment, the at least one polymer that is soluble in the non-aqueous solvent and that has a crystallizable portion is added before the blending operation.

The at least one non-aqueous solvent may be added during the blending or prior to blending, separately or with the at least one polymer that is soluble in the non-aqueous solvent and that has a crystallizable portion.

Without being bound by theory, it appears that blending the composition instead of stirring it according to a conventional process promotes the crystallization of the wax in the form of fine crystals forming small particles. It also appears that this blending breaks up any particle aggregates formed, leading, in the presence of the at least one polymer that is soluble in the non-aqueous solvent medium and that has a crystallizable portion, to a substantially homogeneous dispersion of small wax particles in the non-aqueous solvent medium.

The blending may be performed, for example, in a roll mill comprising two counter-rotating rolls between which is fed the paste, or for instance, in a continuous twin-screw blender, which can allow a paste of consistent quality to be reproducibly obtained.

The conditions under which the blending operation may be performed are described in French Patent Application FR 94/00756, the content of which is incorporated into the present patent application by reference.

In yet another embodiment for preparing the compositions as disclosed herein, the water and/or the at least one water-soluble solvent and the optional additional ingredients are introduced into the starting materials or, optionally, in the course of the blending during the cooling or into the finished composition. This method for preparing the compositions for example, allows the incorporation of heat-sensitive compounds, for instance certain active agents, given that it allows them to be introduced at a temperature that is compatible with their stability and by virtue of the short residence time in the blender.

In further still another embodiment of the present disclosure, the at least one wax used is chosen from microwaxes as defined above. As a result of its formulation in the form of particles, microwaxes may be used directly at a temperature below the melting point. In other words, in this embodiment of preparation of the compositions according to the present disclosure, the microwax particles are dispersed directly in the continuous phase, rather than forming them therein via melting/recrystallization steps.

This wax dispersion step may be performed for example, at a temperature below the melting point of the wax, for instance at room temperature, which is, of course, beneficial in terms of ease of implementation of the preparation process.

The at least one non-aqueous solvent is chosen from those defined above. In one embodiment of the preparation of the compositions as disclosed herein, the water and/or the at least one water-soluble solvent and/or the additional ingredients as defined above may be added, either into the starting materials or into the finished composition.

In another embodiment of the present disclosure, the preparation of the compositions involves both at least one conventional wax and at least one microwax as defined above. In such a case, the conventional wax or the mixture of conventional waxes is generally introduced first, in molten form, into the mixture comprising at least one non-aqueous solvent and at least one polymer that is soluble in the solvent and that has a crystallizable portion, and the mixture thus obtained is then stirred or blended while cooling. The microwax or the mixture of microwaxes is introduced only when the temperature of the mixture comprising the conventional wax is below the melting point of the at least one microwax that has the lowest melting point, such as at room temperature.

In this preparation, the water and/or the at least one water-soluble solvent and the additional ingredients may be added, either into the starting materials or into the finished compositions, or else, when the composition is blended, during the cooling.

An aspect of the present disclosure is also a process for making up keratin fibers, wherein a composition as defined above is applied to the keratin fibers, such as the eyelashes.

The compositions of the present disclosure may for instance, be applied to the eyelashes using a brush or a comb.

The thickening effect of the makeup, using the compositions as disclosed herein, may moreover be reinforced for example, by the choice of the device for applying the composition.

In one embodiment of the present disclosure, for example, when making up the eyelashes, the composition can be applied with a makeup brush as described in French Patent Nos. FR 2701198 and FR 2605505, and European Patent Nos. EP 792603 and EP 663161.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contain certain errors necessarily resulting from the standard deviation found in their respective testing measurements.

The examples that follow are given as non-limiting illustrations of the present disclosure.

### EXAMPLES

Composition Preparation Protocol

a. Preparation of Compositions Comprising Only Waxes in Microparticle Form

The dyestuffs and the gelling agent were dispersed with stirring in a mixture comprising at least one non-aqueous solvent medium and at least one polymer that was soluble in the medium and that had at least one crystallizable portion, heated to a temperature of 45° C. and then cooled to room temperature. The wax in microparticle form and, where appropriate, the remaining ingredients of the composition were then added, with stirring.

The water and the at least one water-soluble solvent were gradually dispersed with stirring.

b. Preparation of Compositions Comprising Both Conventional Waxes and Waxes in Microparticle Form

The dyestuffs and the gelling agent were dispersed with stirring in a mixture comprising at least one non-aqueous solvent medium and at least one polymer that was soluble in the said medium and that had at least one crystallizable portion, heated to a temperature of 45° C. and then cooled to room temperature. The mixture obtained was then heated to 45° C., after which the mixture of conventional waxes preheated until completely melted was gradually added. The mixture thus obtained was allowed to cool to room temperature with stirring. The wax in microparticle form and the remaining ingredients of the composition were then added.

The water and at least one water-soluble solvent were gradually dispersed with stirring.

c. Preparation of the Compositions Using a Continuous Twin-Screw Blender

The preparation was performed in a continuous twin-screw blender such as the “BC-21” model from the company Clextral, and took place under the following conditions:
inlet temperature: 100° C.
outlet temperature: 20° C.
flow rate=3 kg/h
screw speed: 600 rpm.

The premelted waxes were introduced into the top of the blender at the same time as the non-aqueous solvent and the other ingredients, and the mixture was then cooled under continuous twin-screw blending down to the outlet temperature.

Measurement of the Physical Characteristics

The measurement of the solids content was performed according to the protocol described above.

The rheological measurements were performed according to the protocols described above, using a “Haake RheoStress 600®” controlled-stress rheometer under the following conditions:
measuring temperature: 25° C.,
steady stage of 180 seconds at 25° C. before starting the measuring,
stressed sweep from 1 to 10,000 Pa,
measuring frequency: 1 Hz.

### Examples 1 to 3

Three waterproof mascaras were prepared according to the process described in b).

The compositions of these mascaras are presented in Table 1 below, wherein the amounts indicated are in weight percentages and are expressed relative to the total weight of the composition.

TABLE I

| | Example 1 | Example 2 | Example 3 |
|---|---|---|---|
| Beeswax | 8.67 | 8.67 | 13 |
| Carnauba microwax | 12.2 | 16.2 | 18.37 |
| (“MicroCare 350 ®” from | | | |
| Micro Powders) | | | |
| Synthetic microwax | 6.52 | 6.52 | 6.52 |
| (“MicroEase 114S ®” from | | | |
| Micro Powders) | | | |
| Polyvinyl laurate | 0.66 | 0.66 | *** |
| (“Mexomere PP ®” from | | | |
| Chimex) | | | |
| Preserving agent | 0.2 | 0.2 | 0.2 |
| Dye | 5.7 | 5.7 | 5.7 |
| Modified hectorite (“Bentone | 3.6 | 3.6 | 3.6 |
| 38V ®” from Elementis) | | | |
| Propylene carbonate | 1.18 | 1.18 | 1.18 |
| Vinylpyrrolidone/1-eicosene | *** | 5 | *** |
| copolymer (“Antaron V | | | |
| 220 ®” from ISP) | | | |
| Allyl stearate/vinyl acetate | 6.5 | 6.5 | 6.5 |
| copolymer (“Mexomere | | | |
| PQ ®” from Chimex) | | | |
| Ethylenediamine/stearyl | 3 | 3 | 1.5 |
| dilinoleate copolymer | | | |
| (“Uniclear 100 VG ®” from | | | |
| Arizona Chemical) | | | |
| Vinyl acetate/vinyl t-butyl- | 3 | 3 | *** |
| benzoate/crotonic acid | | | |
| copolymer as an aqueous | | | |
| dispersion containing 26.3% | | | |
| AM | | | |
| (“Mexomere PAM ®” from | | | |
| Chimex) | | | |
| Ethyl acrylate/methyl methacrylate | 9 | *** | *** |
| copolymer (80/20) | | | |
| as an aqueous dispersion | | | |
| containing 50% AM | | | |
| (“Daitosol 5000 AD ®” from | | | |
| Daito) | | | |
| Water | *** | *** | 7.32 |
| Isododecane | qs 100 | qs 100 | qs 100 |

The compositions of Examples 1 and 2 comprised water in respective amounts of 6.5% and 2.1% by weight, relative to the total weight of the composition, wherein the water was derived from latices used, i.e. the “Mexomère PAM®” from the company Chimex and the “Daitosol 5000 AD®” from the company Daito Kasey Kogyo.

Various in vitro characteristics of these compositions were studied according to the protocols described above.

The results are given in Table II below.

TABLE II

| | Characteristics | | | |
|---|---|---|---|---|
| | [wax] | | | |
| Compositions | (% m) | D.E. (% m) | [Gp] (Pa) | τc (Pa) |
| Example 1 | 27.4 | 53.0 | 6,000 | 53.7 |
| Example 2 | 31.4 | 57.5 | 7,000 | 60 |
| Example 3 | 37.9 | 56.5 | 13,000 | 70 |

The compositions obtained thus had a high solids content while at the same time maintaining a stiffness modulus that was low enough to allow them to be used under satisfactory conditions.

Moreover, the mascaras obtained applied easily to the eyelashes and allowed a thick makeup result to be obtained on the eyelashes.

### Example 4

A waterproof mascara having the composition below, wherein the amounts indicated are in weight percentages and are expressed relative to the total weight of the composition, was prepared according to the process described in c):

| Example 4 | |
|---|---|
| Tacky wax (“Kester Wax K 82 P ®” from | 32 g |
| the company Koster Keunen) | |
| Dextrin palmitate (“Rheopearl KL ®” from | 5.32 g |
| Chiba Flour) | |
| Vinyl acetate/allyl stearate copolymer | 2.2 g |
| (65/35) (“Mexomere PQ ®” from Chimex) | |
| Polyvinyl laurate (“Mexomere PP ®” from | 0.75 g |
| Chimex) | |
| 12-Hydroxystearic acid oligomer stearate | 0.10 g |
| (“Solsperse 21000 ®” from Avecia) | |
| Silica | 10 g |
| Talc | 0.84 g |
| Pigments | 4.62 g |
| Preserving agents | qs |
| Non-denatured 96° ethyl alcohol | 3 g |
| Isododecane | 40.96 g |

The various in vitro characteristics of this composition were studied according to the protocols described above.

The results are given in Table III below.

TABLE III

Characteristics

| | [wax] | | | |
|---|---|---|---|---|
| Compositions | (% m) | D.E. (% m) | Gp (Pa) | τc (Pa) |
| Example 4 | 32 | 56.0 | 13,000 | 90 |

The composition obtained thus also had a high solids content, allied with a stiffness modulus that was low enough to allow its use under satisfactory conditions.

The mascara applied easily to the eyelashes and allowed a thick and non-tacky makeup result to be obtained on the eyelashes: the eyelashes were well separated.

### Example 5

A waterproof mascara having the composition below, wherein the amounts indicated are in weight percentages and are expressed relative to the total weight of the composition, was prepared according to the process described in c):

| | |
|---|---|
| Microcrystalline wax (“Microwax HW ®” from Paramelt) | 30 g |
| Pigments | 9.24 g |
| Rice starch | 1.68 g |
| Vinyl acetate/allyl stearate copolymer | 4.42 g |
| (“Mexomere PQ ®” from Chimex) | |
| Polyvinyl laurate (“Mexomere PP ®” from Chimex) | 1.5 g |
| Modified hectorite (“Bentone 38V ®” from Elementis) | 0.63 g |
| Propylene carbonate | 0.21 g |
| Ethyl alcohol | 3.0 g |
| 12-hydroxystearic acid oligomer stearate | 0.2 g |
| (“Solsperse 21000 ®” from Avecia) | |
| Preserving agent | 0.4 g |
| Isododecane | 48.72 g |

This composition had a solids content of 48.28% by weight, relative to the total weight of the composition.

### Examples 6 and 7

The following examples were prepared according to the process described in c), wherein the amounts indicated in the table are in weight percentages and are expressed relative to the total weight of the composition.

| | Example 6 | Example 7 |
|---|---|---|
| Tacking wax (“Kester Wax K 82 P ®” from | 24 | 28 |
| KOSTER KEUNEN) | | |
| Synthetic micowax (“MicroEase 114S ®” | 8 | 4 |
| from MICRO POWDERS) | | |
| Polyvinyl laurate (“Mexomere PP ®” from | 0.75 | 0.75 |
| CHIMEX) | | |
| Pigments | 4.6 | 4.6 |
| Propylene carbonate | 0.49 | 0.49 |
| Modified hectorite (“Bentone 38V ®” from | 1.5 | 1.5 |
| ELEMENTIS) | | |
| Allyl stearate/vinyl acetate copolymer | 2.21 | 2.21 |
| (Mexomere PQ ®” from CHIMEX) | | |
| 12-Hydroxystearic acid oligomer stearate | 0.1 | 0.1 |
| (“Solsperse 21000 ®” from AVECIA) | | |
| Silica | 10 | 10 |
| Talc | 0.84 | 0.84 |
| Ethyl alcohol | 7 | 7 |
| Preserving agent | qs | qs |
| Isododecane | Qsp 100 | Qsp 100 |

1. A cosmetic composition for making up keratin fibers, comprising:
(i) a non-aqueous solvent medium,
(ii) at least one wax in an amount of greater than 3% by weight, relative to the total weight of the composition,
(iii) at least one polymer that is soluble in the solvent medium having at least one crystallizable portion, and
(iv) water and/or at least one water-soluble solvent,
wherein the water and/or the at least one water-soluble solvent is present in a total amount of less than or equal to 20% by weight, relative to the total weight of the composition, and the composition has a solids content of greater than 45% by weight, relative to the total weight of the composition.

2. The composition according to claim 1, wherein the solids content ranges from 46% to 80% by weight, relative to the total weight of the composition.

3. The composition according to claim 2, wherein the solids content ranges from 48% to 70% by weight, relative to the total weight of the composition.

4. The composition according to claim 3, wherein the solids content ranges from 50% to 65% by weight, relative to the total weight of the composition.

5. The composition according to claim 1, wherein the at least one polymer has a molar mass ranging from 200 to 1,000,000 g/mol.

6. Composition according to claim 5, wherein the at least one polymer has a molar mass ranging from 500 to 500,000 g/mol.

7. The composition according to claim 6, wherein the at least one polymer has a molar mass ranging from 1,000 to 300,000 g/mol.

8. The composition according to claim 1, wherein the at least one crystallizable portion is present in an amount ranging from 5% to 50% by weight, relative to the total weight of the at least one polymer.

9. The composition according to claim 8, wherein the at least one crystallizable portion is present in an amount ranging from 10% to 50% by weight, relative to the total weight of the at least one polymer.

10. The composition according to claim 9, wherein the at least one crystallizable portion is present in an amount ranging from 30% to 50% by weight, relative to the total weight of the at least one polymer.

11. The composition according to claim 1, wherein the at least one polymer is chosen from:
copolymers of linear and saturated C₁₂ to C₃₀ alkyl(meth)acrylates and of linear C₄ to C₁₀ alkyl(meth)acrylates or of branched and cyclic and/or unsaturated C₄ to C₃₀ alkyl(meth)acrylates;
copolymers of vinyl esters comprising linear and saturated C₁₂ to C₃₀ alkyl groups and of vinyl esters comprising linear C₄ to C₁₀ alkyl groups or of branched or cyclic and/or unsaturated C₄ to C₃₀ alkyl groups;
polycondensates of polyamide type resulting from the condensation between at least one acid chosen from dicarboxylic acids comprising at least 32 carbon atoms, and an alkylenediamine, wherein the polycondensate comprises at least one carboxylic acid end group esterified or amidated with at least one monoalcohol or a monoamine comprising from 12 to 30 linear and saturated carbon atoms; and
lipophilic polyester polycondensates, the ends of which are esterified with a crystallizable acid or alcohol comprising a saturated linear C₁₂ to C₃₀ carbon-based chain.

12. The composition according to claim 11, wherein the at least one polymer is chosen from vinyl acetate/vinyl stearate, vinyl acetate/allyl stearate, vinyl acetate/ethylene, ethylenediamine/stearyl dilinoleate copolymers, hydrogenated butadiene/isoprene block copolymers and poly(12-hydroxystearic acid), wherein at least one of the ends of which is esterified with stearic acid.

13. The composition according to claim 1, wherein the at least one polymer is present in an amount ranging from 0.01% to 30% by weight, relative to the total weight of the composition.

14. The composition according to claim 13, wherein the at least one polymer is present in an amount ranging from 0.1% to 20% by weight, relative to the total weight of the composition.

15. The composition according to claim 14, wherein the at least one polymer is present in an amount ranging from 1% to 10% by weight, relative to the total weight of the composition.

16. The composition according to claim 1, wherein the non-aqueous solvent medium comprises at least one water-insoluble volatile compound that is liquid at room temperature.

17. The composition according to claim 16, wherein the at least one water-insoluble volatile compound is chosen from hydrocarbon-based oils, silicone oils, fluoro oils, and organic solvents.

18. The composition according to claim 17, wherein the hydrocarbon-based oils comprise from 8 to 16 carbon atoms.

19. The composition according to claim 16, wherein the at least one water-insoluble volatile compound is present in the composition in an amount ranging from 5% to 55% by weight, relative to the total weight of the composition.

20. The composition according to claim 19, wherein the at least one water-insoluble volatile compound is present in the composition in an amount ranging from 10% to 50% by weight, relative to the total weight of the composition.

21. The composition according to claim 20, wherein the at least one water-insoluble volatile compound is present in the composition in an amount ranging from 20% to 45% by weight, relative to the total weight of the composition.

22. The composition according to claim 1, wherein the at least one water-soluble solvent is chosen from lower monoalcohols comprising from 1 to 5 carbon atoms, glycols comprising from 2 to 8 carbon atoms, C₃ and C₄ ketones, and C₂ to C₄ aldehydes.

23. The composition according to claim 1, wherein the total amount of water and/or at least one water-soluble solvent is greater than or equal to 0.5% by weight, relative to the total weight of the composition.

24. The composition according to claim 23, wherein the total amount of water and/or at least one water-soluble solvent ranges from 1% to 18% by weight, relative to the total weight of the composition.

25. The composition according to claim 24, wherein the total amount of water and/or at least one water-soluble solvent ranges from 2% to 15% by weight, relative to the total weight of the composition.

26. The composition according to claim 1, wherein the at least one wax is chosen from waxes that are solid and rigid at room temperature with a melting point of greater than or equal to 30° C.

27. The composition according to claim 26, wherein the at least one wax is chosen from waxes that are solid and rigid at room temperature with a melting point of greater than or equal to 45° C.

28. The composition according to claim 27, wherein the at least one wax is chosen from waxes that are solid and rigid at room temperature with a melting point of greater than or equal to 55° C.

29. The composition according to claim 1, wherein the at least one wax is chosen from hydrocarbon-based waxes; the waxes obtained by catalytic hydrogenation of animal and plant oils comprising linear and branched C₈-C₃₂ fatty chains; and the waxes obtained by hydrogenation of castor oil esterified with cetyl alcohol.

30. The composition according to claim 29, wherein the hydrocarbon-based waxes are chosen from beeswax, lanolin wax, Chinese insect waxes, sumac wax, paraffins, polyethylene waxes, waxy copolymers and esters thereof.

31. The composition according to claim 29, wherein the waxes obtained by catalytic hydrogenation of animal and plant oils comprising linear and branched C₈-C₃₂ fatty chains are chosen from trans-isomerized partially hydrogenated jojoba oil, hydrogenated sunflower oil, hydrogenated castor oil, hydrogenated coconut oil, hydrogenated lanolin oil and bis(1,1,1-trimethylolpropane)tetrastearate.

32. The composition according to claim 1, wherein the at least one wax is chosen from waxes with a tack of greater than or equal to 0.7 N.s and a hardness of less than or equal 3.5 MPa.

33. The composition according to claim 32, wherein the at least one wax is chosen from waxes with a tack of greater than or equal to 1 N.s.

34. The composition according to claim 1, wherein the at least one wax is chosen from C₂₀-C₄₀ alkyl(hydroxystearyloxy)stearates.

35. The composition according to claim 1, wherein the at least one wax is chosen from waxes with a starting melting point of greater than or equal to 45° C.

36. The composition according to claim 35, wherein the at least one wax is chosen from waxes with a starting melting point of greater than or equal to 50° C.

37. The composition according to claim 36, wherein the at least one wax is chosen from waxes with a starting melting point of greater than or equal to 55° C.

38. The composition according to claim 37, wherein the at least one wax is chosen from waxes with a starting melting point of greater than or equal to 60° C.

39. The composition according to claim 1, wherein the at least one wax is chosen from carnauba wax, rice bran wax, candelilla wax, ouricurry wax, montan wax, ozokerite, waxes obtained by Fisher-Tropsch synthesis, hydrogenated jojoba oil, bis(1,1,1-trimethylolpropane)tetrabehenate, waxes obtained by catalytic hydrogenation of olive oil esterified with stearyl alcohol, microcrystalline waxes and polyethylene waxes.

40. The composition according to claim 1, wherein the total wax content ranges from 10% to 70% by weight, relative to the total weight of the composition.

41. The composition according to claim 40, wherein the total wax content ranges from 15% to 65% by weight, relative to the total weight of the composition.

42. The composition according to claim 41, wherein the total wax content ranges from 20% to 60% by weight, relative to the total weight of the composition.

43. The composition according to claim 42, wherein the total wax content ranges from 25% to 55% by weight, relative to the total weight of the composition.

44. The composition according to claim 1, further comprising at least one film-forming polymer.

45. The composition according to claim 1, further comprising at least one dyestuff.

46. The composition according to claim 1, further comprising at least one filler.

47. The composition according to claim 1, further comprising at least one cosmetically acceptable additive chosen from antioxidants, preserving agents, fragrances, neutralizers, plasticizers, fibers, gelling agents and cosmetic active agents.

48. The composition according to claim 1, said composition having a plateau modulus of rigidity Gp of less than or equal to 30,000 Pa.

49. The composition according to claim 48, wherein the plateau modulus of rigidity Gp is less than or equal to 28,000 Pa.

50. The composition according to claim 49, wherein the plateau modulus of rigidity Gp is less than or equal to 25,000 Pa.

51. The composition according to claim 50, wherein the plateau modulus of rigidity Gp is less than or equal to 20,000 Pa.

52. The composition according to claim 1, said composition having a flow threshold τ_{c} measured by oscillatory rheology (γ=1 Hz) ranging from 10 to 200 Pa.

53. A process for preparing a cosmetic composition comprising:
(i) a non-aqueous solvent medium,
(ii) at least one wax in an amount of greater than 3% by weight, relative to the total weight of the composition,
(iii) at least one polymer that is soluble in the solvent medium having at least one crystallizable portion, and
(iv) water and/or at least one water-soluble solvent,
wherein the water and/or the at least one water-soluble solvent is present in a total amount of less than or equal to 20% by weight, relative to the total weight of the composition, and the composition has a solids content of greater than 45% by weight, relative to the total weight of the composition.
the process comprising the continuous blending of the at least one wax and the at least one polymer by passing from a temperature above the melting point of the at least one wax to room temperature with continuous cooling.

54. The process according to claim 53, wherein the blending takes place in a continuous twin-screw blender.

55. The process according to claim 53, wherein at least one non-aqueous solvent is added either prior to the blending or during the blending.

56. A process for preparing a cosmetic composition comprising:
(i) a non-aqueous solvent medium,
(ii) at least one wax in an amount of greater than 3% by weight, relative to the total weight of the composition,
(iii) at least one polymer that is soluble in the solvent medium having at least one crystallizable portion, and
(iv) water and/or at least one water-soluble solvent,
wherein the water and/or the at least one water-soluble solvent is present in a total amount of less than or equal to 20% by weight, relative to the total weight of the composition, and the composition has a solids content of greater than 45% by weight, relative to the total weight of the composition.
the process comprising dispersing at least one wax in the form of particles ranging from 0.5 μm to 30 μm in size, in a mixture comprising the at least one non-aqueous solvent and the at least one polymer, wherein the mixture is at a temperature below the melting point of the at least one wax in particle form.

57. The process according to claim 56, wherein the dispersing is performed at room temperature.

58. The process according to claim 56, wherein the at least one wax in in the form of particles ranging from 1 to 20 μm.

59. The process according to claim 56, wherein the at least one wax is chosen from carnauba wax, synthetic wax, waxes comprising a mixture of carnauba wax and of polyethylene wax, waxes comprising a mixture of carnauba wax and synthetic wax, polyethylene waxes and polytetrafluoroethylene waxes.

60. The process according to claim 56, wherein the at least one wax is chosen from waxes that are solid and rigid at room temperature with a melting point of greater than or equal to 30° C., wherein the at least one wax is introduced in molten form before blending into the mixture and the mixture is then allowed to cool with stirring or blending down to a temperature at least below the melting point of the at least one wax in particle form.

61. The process according to claim 56, wherein the non-aqueous solvent comprises at least one water-insoluble volatile compound that is liquid at room temperature.

62. The process according to claim 61, wherein the at least one water-insoluble volatile compound is chosen from hydrocarbon-based oils, silicone oils, fluoro oils, and organic solvents.

63. The process according to claim 53, wherein the at least one polymer has a molar mass ranging from 200 to 1,000,000 g/mol.

64. The process according to claim 53, wherein the at least one crystallizable portion is present in an amount ranging from 5% to 50% by weight, relative to the total weight of the at least one polymer.

65. The process according to claim 53, wherein the at least one polymer is chosen from:
copolymers of linear and saturated C₁₂ to C₃₀ alkyl(meth)acrylates and of linear C₄ to C₁₀ alkyl(meth)acrylates or of branched and cyclic and/or unsaturated C₄ to C₃₀ alkyl(meth)acrylates;
copolymers of vinyl esters comprising linear and saturated C₁₂ to C₃₀ alkyl groups and of vinyl esters comprising linear C₄ to C₁₀ alkyl groups or of branched or cyclic and/or unsaturated C₄ to C₃₀ alkyl groups;
polycondensates of polyamide type resulting from the condensation between at least one acid chosen from dicarboxylic acids comprising at least 32 carbon atoms, and an alkylenediamine, wherein the polycondensate comprises at least one carboxylic acid end group esterified or amidated with at least one monoalcohol or a monoamine comprising from 12 to 30 linear and saturated carbon atoms; and
lipophilic polyester polycondensates, the ends of which are esterified with a crystallizable acid or alcohol comprising a saturated linear C₁₂ to C₃₀ carbon-based chain.

66. The process according to claim 53, wherein the at least one polymer is present in an amount ranging from 0.01% to 30% by weight, relative to the total weight of the composition.

67. The process according to claim 56, wherein the at least one polymer that is soluble in the non-aqueous solvent and has at least one crystallizable portion has a molar mass ranging from 200 to 1,000,000 g/mol.

68. The process according to claim 56, wherein the at least one crystallizable portion is present in an amount ranging from 5% to 50% by weight, relative to the total weight of the at least one polymer.

69. The process according to claim 56, wherein the at least one polymer is chosen from:
copolymers of linear and saturated C₁₂ to C₃₀ alkyl(meth)acrylates and of linear C₄ to C₁₀ alkyl(meth)acrylates or of branched and cyclic and/or unsaturated C₄ to C₃₀ alkyl(meth)acrylates;
copolymers of vinyl esters comprising linear and saturated C₁₂ to C₃₀ alkyl groups and of vinyl esters comprising linear C₄ to C₁₀ alkyl groups or of branched or cyclic and/or unsaturated C₄ to C₃₀ alkyl groups;
polycondensates of polyamide type resulting from the condensation between at least one acid chosen from dicarboxylic acids comprising at least 32 carbon atoms, and an alkylenediamine, wherein the polycondensate comprises at least one carboxylic acid end group esterified or amidated with at least one monoalcohol or a monoamine comprising from 12 to 30 linear and saturated carbon atoms; and
lipophilic polyester polycondensates, the ends of which are esterified with a crystallizable acid or alcohol comprising a saturated linear C₁₂ to C₃₀ carbon-based chain.

70. The process according to claim 56, wherein the at least one polymer is present in an amount ranging from 0.01% to 30% by weight, relative to the total weight of the composition.

71. The process according to claim 53, wherein the prepared cosmetic composition has a solids content of greater than 45% by weight, relative to the total weight of the composition.

72. The process according to claim 71, wherein the prepared cosmetic composition has a solids content of greater than or equal to 50% by weight, relative to the total weight of the composition.

73. The process according to claim 56, wherein the prepared cosmetic composition has a solids content of greater than or equal to 45% by weight, relative to the total weight of the composition.

74. The process according to claim 73, wherein the prepared cosmetic composition has a solids content of greater than or equal to 50% by weight, relative to the total weight of the composition.

75. The process according to claim 53, wherein the prepared cosmetic composition has a plateau modulus of rigidity of less than or equal to 30,000 Pa.

76. The process according to claim 75, wherein the prepared cosmetic composition has a plateau modulus of rigidity of less than or equal to 20,000 Pa.

77. The process according to claim 56, wherein the prepared cosmetic composition has a plateau modulus of rigidity of less than or equal to 30,000 Pa.

78. The process according to claim 77, wherein the prepared cosmetic composition has a plateau modulus of rigidity of less than or equal to 20,000 Pa.

79. A method for making up keratin fibers, comprising applying to the keratin fibers, a composition comprising
(i) a non-aqueous solvent medium,
(ii) at least one wax in an amount of greater than 3% by weight, relative to the total weight of the composition,
(iii) at least one polymer that is soluble in the solvent medium having at least one crystallizable portion, and
(iv) water and/or at least one water-soluble solvent,
wherein the water and/or the at least one water-soluble solvent is present in a total amount of less than or equal to 20% by weight, relative to the total weight of the composition, and the composition has a solids content of greater than 45% by weight, relative to the total weight of the composition.

80. The method according to claim 79, wherein the keratin fibers are eyelashes.
